(19) United States
(12) Patent Application Publication
(10) Pub. No.: US 2007/0021711 A1
(43) Pub. Date: Jan. 25, 2007

(54) Iontophoresis device controlling administration amount and administration period of plurality of drugs

(75) Inventors: Akihiko Matsumura, Shibuya-ku (JP); Takehiko Matsumura, Shibuya-ku (JP); Mizuo Nakayama, Shibuya-ku (JP); Hidero Akiyama, Shibuya-ku (JP); Tsutomu Shibata, Shibuya-ku (JP)

Correspondence Address: SEED INTELLECTUAL PROPERTY LAW GROUP PLLC, 701 FIFTH AVE, SUITE 5400, SEATTLE, WA, 98104, US

(73) Assignee: Transcutaneous Technologies, Inc., Shibuya-ku (JP)

(21) Appl. No.: 11/473,515

(22) Filed: Jun. 22, 2006

(30) Foreign Application Priority Data
Jun. 23, 2005 (JP) 2005-183404

(51) Int. Cl. A61N 1/30

(52) U.S. Cl. 604/20

(57) ABSTRACT

An iontophoresis device capable of administering a plurality of drugs to a living body while controlling the administration amounts and the administration periods thereof is described. The iontophoresis device may comprise: a power source device; a drug administration unit connected to the power source device and including at least two electrode structures that hold an ionic drug; and a current control unit that individually controls currents flowing to the electrode structures. A predetermined amount of the ionic drug may be released from each of the electrode structures to be administered transdermally to a living body in a predetermined period of time according to a current flowing from the current control unit.

### BACKGROUND OF THE INVENTION

1. Field of the Invention

The present disclosure relates to transdermal drug delivery, a technique of transdermally administering various kinds of ionic drugs by iontophoresis, and in particular, to an iontophoresis device for administering a plurality of drugs while controlling the administration amount and administration period of each drug.

2. Description of the Related Art

A method of introducing (delivering) into the body an ionic drug placed on a skin or mucosa surface (hereinafter referred to as “skin”) of a predetermined site of a living body through the skin by giving the skin an electromotive force sufficient to drive such an ionic drug, is called iontophoresis (iontophorese, ion introduction method, ion permeation therapy) (see JP 63-35266 A).

For example, positively charged ions may be driven (transported) into the skin on an anode side (positive electrode) of an iontophoresis device. On the other hand, negatively charged ions may be driven (transported) into the skin on a cathode side (negative electrode) of the iontophoresis device.

A variety of iontophoresis devices have been proposed (see, for example, JP 63-35266 A, JP 04-297277 A, JP 2000-229128 A, JP 2000-229129 A, JP 2000-237327 A, JP 2000-237328 A, and WO 03/037425 A1).

Conventional iontophoresis devices may, in principle, be suited to transdermally administering one drug. However, it may be necessary to administer a plurality of drugs while controlling the administration period and administration amount of each drug in order to effect appropriate treatment on a patient, depending upon the disease, patient condition, and the like.

Thus, it may be important to make it possible in an iontophoresis device to administer a plurality of drugs to a living body while controlling their administration amounts and administration periods.

### BRIEF SUMMARY OF THE INVENTION

In view of the above-mentioned issues, in at least one embodiment an iontophoresis device may be capable of administering a plurality of drugs to a living body while controlling the administration amount and the administration period of time for each drug.

The iontophoresis device described above may comprise: a power source device; a drug administration unit connected to the power source device and comprising at least two electrode structures that hold an ionic drug; and a current control unit that individually controls currents flowing to the electrode structures, wherein a predetermined amount of the ionic drug is released from each of the electrode structures to be administered transdermally to a living body in a predetermined period of time according to a current flowing from the current control unit.

Further, in at least one embodiment, the drug administration unit comprises at least two first electrode structures that hold an ionic drug, and at least one second electrode structure that does not hold an ionic drug and acts as a counter electrode to the first electrode structures.

In at least one embodiment the first electrode structure may comprise: an electrode having the same polarity as that of a drug component of the ionic drug in the first electrode structure, the electrode being connected to the power source device; an electrolyte solution holding portion impregnated with an electrolyte solution, the electrolyte solution holding portion being placed adjacent to the electrode; an ion exchange membrane that selectively passes ions having a polarity opposite to that of a charged ion of the ionic drug, the ion exchange membrane being placed adjacent to the electrolyte solution holding portion; a drug holding portion impregnated with an ionic drug, the drug holding portion being placed adjacent to the ion exchange membrane; and an ion exchange membrane that selectively passes ions having the same polarity as that of a charged ion of the ionic drug, the ion exchange membrane being placed adjacent to the drug holding portion, and the second electrode structure may comprise: an electrode having a polarity opposite to that of the electrode of the first electrode structure; an electrolyte solution holding portion impregnated with an electrolyte solution, the electrolyte solution holding portion being placed adjacent to the electrode; and an ion exchange membrane that selectively passes ions having a polarity opposite to that of a charged ion of the ionic drug in the first electrode structure, the ion exchange membrane being placed adjacent to the electrolyte solution holding portion.

In at least one embodiment the second electrode structure may comprise: an electrode having a polarity opposite to that of the electrode in the first electrode structure; an electrolyte solution holding portion impregnated with an electrolyte solution, the electrolyte solution holding portion being placed adjacent to the electrode; an ion exchange membrane that selectively passes ions having the same polarity as that of a charged ion of the ionic drug in the first electrode structure, the ion exchange membrane being placed adjacent to the electrolyte solution holding portion; an electrolyte solution holding portion impregnated with an electrolyte solution, the electrolyte solution holding portion being placed adjacent to the ion exchange membrane; and an ion exchange membrane that selectively passes ions having a polarity opposite to that of a charged ion of the ionic drug in the first electrode structure, the ion exchange membrane being placed adjacent to the electrolyte solution holding portion.

In at least one embodiment the drug administration unit may comprise at least one first electrode structure that holds an ionic drug and at least one second electrode structure that holds an ionic drug as a counter electrode to the first electrode structure.

In addition, in at least one embodiment the first electrode structure may comprise: an electrode having the same polarity as that of a drug component of the ionic drug in the first electrode structure, the electrode being connected to the power source device; an electrolyte solution holding portion impregnated with an electrolyte solution, the electrolyte solution holding portion being placed adjacent to the electrode; an ion exchange membrane that selectively passes ions having a polarity opposite to that of a charged ion of the ionic drug in the first electrode structure, the ion exchange membrane being placed adjacent to the electrolyte solution holding portion; a drug holding portion impregnated with an ionic drug, the drug holding portion being placed adjacent to the ion exchange membrane; and an ion exchange membrane that selectively passes ions having the same polarity as that of a charged ion of the ionic drug in the first electrode structure, the ion exchange membrane being placed adjacent to the drug holding portion, and the second electrode structure may comprise: an electrode having a polarity opposite to that of the electrode of the first electrode structure; an electrolyte solution holding portion impregnated with an electrolyte solution, the electrolyte solution holding portion being placed adjacent to the electrode; an ion exchange membrane that selectively passes ions having a polarity opposite to that of a charged ion of the ionic drug in the second electrode structure, the ion exchange membrane being placed adjacent to the electrolyte solution holding portion; a drug holding portion impregnated with an ionic drug, the drug holding portion being placed adjacent to the ion exchange membrane; and an ion exchange membrane that selectively passes ions having the same polarity as that of a charged ion of the ionic drug in the second electrode structure, the ion exchange membrane being placed adjacent to the drug holding portion.

Further, in at least one embodiment the drug administration unit may be configured integrally.

In at least one embodiment the current control unit may comprise a load resistor provided between the electrode structure and the power source device, a current detecting part detecting a current flowing to the load resistor, and a feedback control part allowing a controlled current to flow to the electrode structure.

Additionally, in at least one embodiment a method of operating the iontophoresis device may comprise:

placing the drug administration unit on a skin surface of a living body;

passing a current through the drug administration unit with the power source device;

individually controlling currents flowing to the electrode structures with the current control unit to allow the controlled current to flow to the electrode structures; and

releasing a predetermined amount of the ionic drug from each of the electrode structures in a predetermined period of time.

The current control unit that individually controls currents flowing to a plurality of electrode structures each holding an ionic drug may be used, and a predetermined amount of the ionic drug may be released from each of the electrode structures in a predetermined period of time (i.e., in a predetermined period of time) at a predetermined timing, according to a current flowing from the current control unit. Therefore, a plurality of drugs may be administered to a patient while controlling the administration amounts and the administration periods of time for the plurality of drugs. Furthermore, the administration amount and administration period of time may be controlled independently with respect to the plurality of electrode structures. This may make it possible to adjust the administration amount and administration period of time for a particular drug, allowing treatment appropriate for the specific condition of a patient to be performed. Furthermore, by selecting drugs expected to have a synergistic effect, the plurality of ionic drugs may be administered appropriately to a patient for effective treatment.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a bottom view of an iontophoresis device according to an embodiment of the present invention;

FIG. 2 is a cross-sectional view of a drug administration unit in an iontophoresis device according to an embodiment of the present invention;

FIG. 3 is a cross-sectional view of a drug administration unit in an iontophoresis device according to an embodiment of the present invention; and

FIG. 4 is a circuit diagram of an iontophoresis device according to an embodiment of the present invention.

### DETAILED DESCRIPTION OF THE INVENTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with iontophoresis devices, controllers, voltage or current sources and/or membranes have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

The headings provided herein are for convenience only and do not interpret the scope or meaning of the embodiments.

As described above, an iontophoresis device according to one embodiment of the present invention may comprise: a power source device; a drug administration unit connected to the power source device and comprising at least two electrode structures that hold an ionic drug; and a current control unit for individually controlling currents flowing to the electrode structures, wherein a predetermined amount of the ionic drug is released to be transdermally administered to a living body from each of the electrode structures in a predetermined period of time, according to a current flowing from the current control unit.

Embodiments of the present invention are described with reference to specific examples illustrated in the drawings.

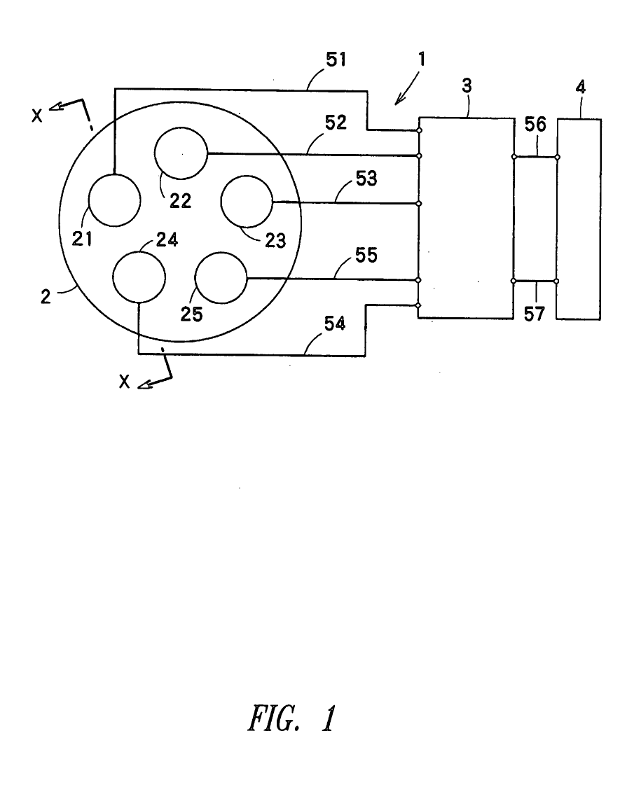

FIG. 1 is a bottom view of an iontophoresis device 1. The iontophoresis device 1 comprises a drug administration unit 2 to be placed on the skin of a living body, a current control unit 3, and a power source device 4. The drug administration unit 2 includes a plurality of electrode structures, among which first electrode structures 21, 22, and 23 are electrically coupled to the current control unit 3 via conductors 51, 52, and 53, respectively, and second electrode structures 24 and 25, which are counter electrodes of the first electrode structures 21, 22, and 23, are electrically coupled to the current control unit 3 via conductors 54 and 55, respectively. The current control unit 3 is connected to the power source device 4 through wirings 56 and 57.

The electrode structures 21, 22, 23, 24, and 25 in the drug administration unit 2 are collected in one package to be configured integrally in the example described above, but they may be configured separately from each other. Alternatively, only a portion of the plurality of electrode structures may be configured integrally.

Furthermore, although the drug administration unit 2, the current control unit 3, and the power source device 4 are provided separately in the example described above, the drug administration unit 2, the current control unit 3, and the power source device 4 may also be configured integrally by using a button battery as the power source device 4 and configuring the current control unit 3 as a miniaturized integrated circuit, for example.

In addition, an ionic drug may be held in all or in a portion of the electrode structures of the drug administration unit 2.

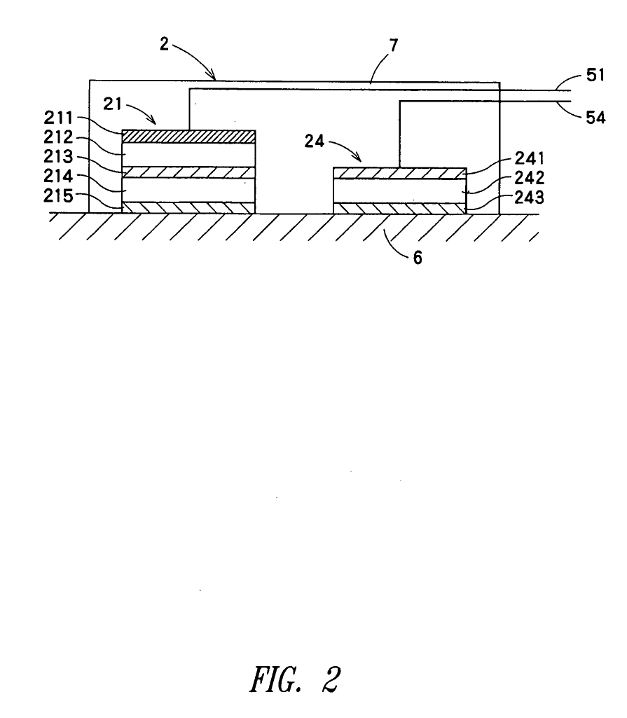

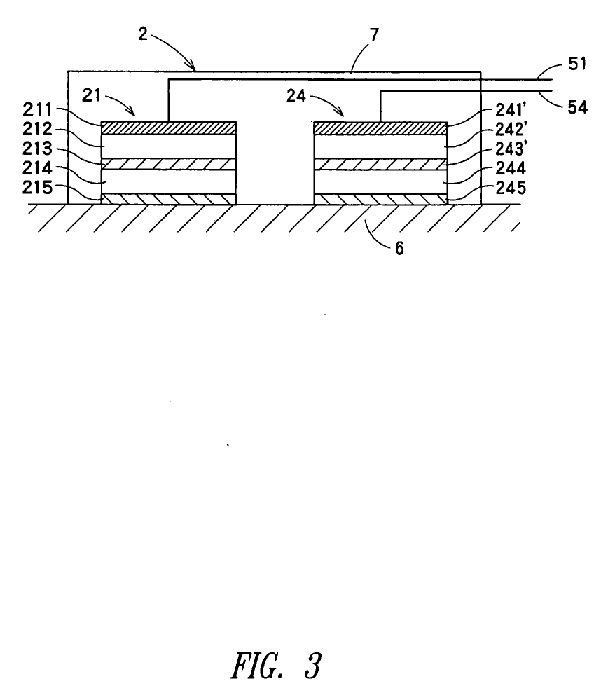

Referring to FIGS. 2 and 3, a specific electrode structure configuration is used to explain cases where the first electrode structure holds an ionic drug, and the second electrode structure does not hold an ionic drug, and where both the first electrode structure and the second electrode structure hold an ionic drug.

FIGS. 2 and 3 are cross sectional views of the drug administration unit 2 in FIG. 1 taken along a line X-X′. The drug administration unit 2 is placed on a skin 6. The electrode structures 21 and 24 are backed by one package 7.

Referring to FIG. 2, a specific example is shown in which the first electrode structure 21 holds an ionic drug, and the second electrode structure 24 does not hold an ionic drug.

The first electrode structure 21 comprises: an electrode 211 having the same polarity as that of a drug component of the ionic drug in the first electrode structure 21, the electrode 211 being electrically coupled to the power source device 4 via the conductor 51; an electrolyte solution holding portion 212 impregnated with an electrolyte solution, the electrolyte solution holding portion 212 being placed adjacent to the electrode 211; an ion exchange membrane 213 that selectively passes ions having a polarity opposite to that of a charged ion of the ionic drug, the ion exchange membrane 213 being placed adjacent to the electrolyte solution holding portion 212; a drug holding portion 214 impregnated with an ionic drug, the drug holding portion 214 being placed adjacent to the ion exchange membrane 213; and an ion exchange membrane 215 that selectively passes ions having the same polarity as that of a charged ion of the ionic drug, the ion exchange membrane 215 being placed adjacent to the drug holding portion 214. The second electrode structure 24 electrically coupled to the power source device 4 via the conductor 54 comprises: an electrode 241 having a polarity opposite to that of the electrode 211 in the first electrode structure 21; an electrolyte solution holding portion 242 impregnated with an electrolyte solution, the electrolyte solution holding portion 242 being placed adjacent to the electrode 241; and an ion exchange membrane 243 that selectively passes ions having a polarity opposite to a charged ion of the ionic drug in the first electrode structure 21, the ion exchange membrane 243 being placed adjacent to the electrolyte solution holding portion 242.

Referring to FIG. 3, a specific example of the second electrode structure 24 not holding an ionic drug is described.

The first electrode structure 21 is configured similarly to that shown in FIG. 2. However, the second electrode structure 24 comprises: an electrode 241′ having a polarity opposite to that of the electrode 211 in the first electrode structure 21; an electrolyte solution holding portion 242′ impregnated with an electrolyte solution, the electrolyte solution holding portion 242′ being placed adjacent to the electrode 241′; an ion exchange membrane 243′ that selectively passes ions having the same polarity as that of a charged ion of the ionic component in the first electrode structure 21, the ion exchange membrane 243′ being placed adjacent to the electrolyte solution holding portion 242′; an electrolyte solution holding portion 244 impregnated with an electrolyte solution, the electrolyte solution holding portion 244 being placed adjacent to the ion exchange membrane 243′; and an ion exchange membrane 245 that selectively passes ions having a polarity opposite to that of a charged ion of the ionic drug in the first electrode structure 21, the ion exchange membrane 245 being placed adjacent to the electrolyte solution holding portion 244.

Referring to FIG. 3, a specific example in which the first electrode structure 21 holds an ionic drug and the second electrode structure 24 holds an ionic drug is described. The first electrode structure and the second electrode structure are opposite polarity electrodes in this example, so that the ionic drug in the first electrode structure and the ionic drug in the second electrode structure are ionized to opposite polarities. The drug holding portion 244 impregnated with an ionic drug ionized to a polarity opposite to that of the ionic drug in the first electrode structure 21 is used in place of the electrolyte solution holding portion 244. The remaining configuration is the same as that of the specific example described above where the second electrode structure 24 does not hold an ionic drug.

More specifically, when the electrode structure 24 holds an ionic drug, the electrode structure 24 comprises: the electrode 241′ having a polarity opposite to that of the electrode 211 of the first electrode structure 21; an electrolyte solution holding portion 242′ impregnated with an electrolyte solution, the electrolyte solution holding portion 242′ being placed adjacent to the electrode 241′; an ion exchange membrane 243′ that selectively passes ions having a polarity opposite to that of a charged ion of the ionic drug in the second electrode structure 24, the ion exchange membrane 243′ being placed adjacent to the electrolyte solution holding portion 242′; a drug holding portion 244 impregnated with an ionic drug, the drug holding portion 244 being placed adjacent to the ion exchange membrane 243′; and an ion exchange membrane 245 that selectively passes ions having the same polarity as that of a charged ion of the ionic drug in the second electrode structure 24; the ion exchange membrane 245 being placed adjacent to the drug holding portion 244.

When a current is allowed to pass through the electrode structures 21 and 24 that hold an ionic drug, the ionic drug moves to an opposite side of the electrodes 211 and 241′ by electrophoresis owing to an electric field, and is administered to the skin via the ion exchange membranes 215 and 245. The ion exchange membranes 213 on the electrode 211 side and the ion exchange membrane 243′ on the electrode 241′ side selectively pass ions having a polarity opposite to that of a charged ion of the ionic drug. This prevents the ionic drug from moving to the electrode 211 side and the electrode 241′ side. The ion exchange membranes 215 and 245′ placed in transmitting relation with the skin selectively pass ions having the same polarity as that of a charged ion of the ionic drug. Therefore, the ionic drug may be released efficiently, and the ionic drug may be administered to the skin at a high transport efficiency. Damage to the skin based on an electrochemical reaction may thus be reduced, making it possible to administer the ionic drug more safely.

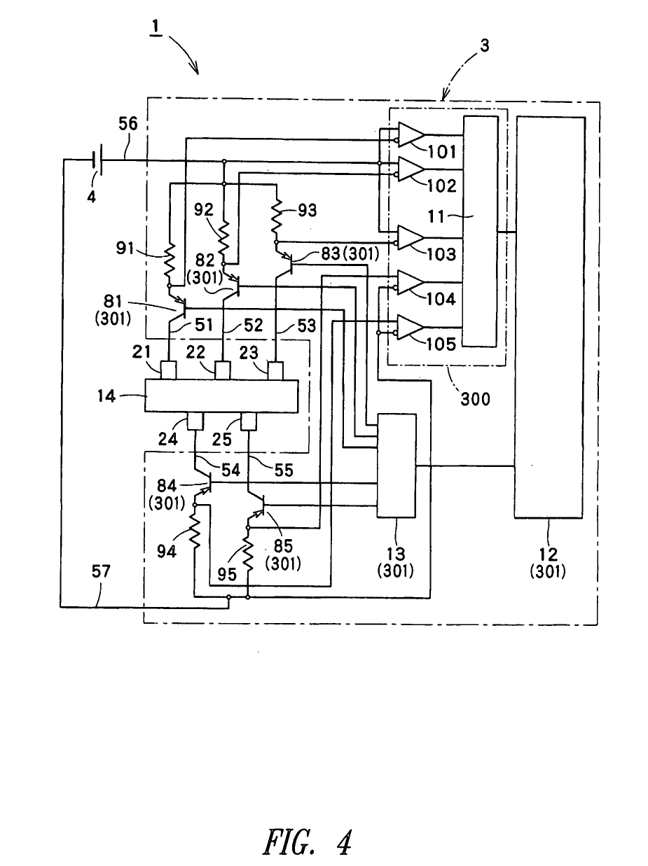

Referring to FIG. 4, a specific example of the current control unit of the iontophoresis device 1 is described next. The iontophoresis device 1 may enable the release of a predetermined amount of ionic drug in a predetermined period of time owing to the circuit shown in FIG. 4, and furthermore, may enable a current with a predetermined value to flow to each electrode structure holding an ionic drug, irrespective of the skin impedance and changes over time.

As shown in FIG. 4, the current control unit 3 in the iontophoresis device 1 comprises: load resistors 91, 92, 93, 94, and 95 provided between the electrode structures 21, 22, 23, 24, and 25, respectively, and the power source device 4; a current detecting portion 300 that detects currents flowing to the load resistors 91, 92, 93, 94, and 95; and a feedback control portion 301 that allows controlled currents to flow to the electrode structures 21, 22, 23, 24, and 25 according to outputs from the current detecting portion 300.

The current detecting portion 300 comprises: current detecting circuits 101, 102, 103, 104, and 105 that detect currents flowing to the load resistors 91, 92, 93, 94, and 95, respectively, and an A/D converter 11 that converts the outputs from the current detecting circuits 101, 102, 103, 104, and 105 to digital signals, and outputs the digital signals to the feedback control portion 301.

Furthermore, the feedback control portion 301 comprises: a CPU 12 that outputs a feedback signal to the electrode structures 21, 22, 23, 24, and 25 according to output from the current detecting portion 300; a D/A converter 13 that converts a feedback signal to an analog signal; and transistors 81, 82, 83, 84, and 85 provided between the electrode structures 21, 22, 23, 24, and 25 and the load resistors 91, 92, 93, 94, and 95, respectively, allowing controlled currents to flow to the electrode structures 21, 22, 23, 24, and 25 according to output from the D/A converter 13. Emitters of the transistors 81, 82, 83, 84, and 85 are coupled to the load resistors 91, 92, 93, 94, and 95, respectively, bases thereof are coupled to the D/A converter 13, and collectors thereof are coupled to the electrode structures 21, 22, 23, 24, and 25, respectively.

A differential amplifier may be used in each of the current detecting circuits 101, 102, 103, 104, and 105. Differential amplifiers are capable of detecting a voltage value across each of the load resistors 91, 92, 93, 94, and 95. Current values may be computed from the voltage values and the resistance of each of the load resistors.

Furthermore, the load resistors 91, 92, 93, 94, and 95 may preferably have fixed resistances. The resistance of the fixed resistor can be appropriately set according to a previously determined value of current flowing to each electrode structure. Considering factors such as the influence on the working state of the iontophoresis device, the load resistance may preferably be set to a value of 10 Ω or less.

Operation of the iontophoresis device 1 is described with reference to FIG. 4.

First, the current detecting circuits 101, 102, 103, 104, and 105 detect currents flowing to the load resistors 91, 92, 93, 94, and 95, respectively, from the power source device 4. Signals corresponding to the detected currents are transmitted to the CPU 12 via the A/D converter 11. Next, the CPU 12 responds to the signals from the A/D converter 11 by performing predetermined data processing and transmits a feedback signal to the D/A converter 13. The D/A converter 13 allows the current responding to the feedback signal from the CPU 12 to the transistor. Predetermined amounts of the ionic drug are released from the electrode structures 21, 22, 23, 24, and 25, in a predetermined period of time, based on the currents flowing from the transistors 81, 82, 83, 84, and 85, respectively. The ionic drug is thus administered to the living body 14 transdermally.

The CPU 12 contains a predetermined algorithm, performs data processing based on the algorithm, and outputs a feedback signal to release a predetermine amount of ionic drug in a predetermined period of time in each electrode structure. Parameters such as the order in which current flows to each electrode structure, periods of time thereof, and combinations of respective electrode structures can be altered by appropriately changing the program in the CPU.

Furthermore, the CPU 12 can perform control so that predetermined current values flow to each electrode structure irrespective of skin impedance and changes thereof over time. Such control can be performed, for example, according to the following multivariate control scheme.

It is assumed that the actual measured values of currents through the respective load resistors 91, 92, 93, 94, and 95 are I91, I92, I93, I94, and I95, respectively, and that actual the measured voltages values thereof are V91, V92, V93, V94, and V95, respectively. It is also assumed that a current vector Ii=(I91, I92, I93, I94, I95), a voltage vector Vi=(V91, V92, V93, V94, V95), and an Expression (1) Ii=MA+MB×Vi is true. MA represents a matrix expressing an internal state of a system, with no dependence upon Vi, and MB represents a matrix expressing skin resistance and internal resistance in an iontophoresis device with respect to the ionic drug. MA and MB are found from Ii and Vi measured successively by the current detecting circuit and Expression (1). An Expression (2) Vi=Inv(MB) (Ii−MA) may be found from MA, MB, and Expression (1). A control voltage Vi for the current value Ii may then be calculated. The CPU 12 outputs a feedback signal to attain the control voltage Vi thus determined, performing control so that a current with a predetermined value flows to each electrode structure. Thus, according to one embodiment, the current control unit in the iontophoresis device performs control so that a current with a predetermined value flows to the electrode structure.

Furthermore, the following conditions are adopted as preferable energization conditions in the iontophoresis device 1:
(1) Constant current, specifically current from 0.1 to 0.5 mA/cm2, preferably from 0.1 to 0.3 mA/cm2
(2) Safe voltage condition to attain the constant current condition, specifically, 50 V or less, preferably 30 V or less

The total number of electrode structures and the combination of the first electrode structures and the second electrode structures are not limited to the number shown in the above specific example. The embodiments may be practiced when the number of total electrode structures, the number of first electrode structures, and/or the number of second electrode structures is suitably changed. For example, an increase/decrease of the number of electrode structures can be performed by increasing/decreasing the number of transistors, load resistors, current detecting circuits, or the like in FIG. 4 by a required amount.

In addition, although the ionic drugs held by the respective electrode structures are preferably different kinds of drugs, a part of the plurality of electrode structures may hold the same kind of drug, depending upon the form of treatment to be performed.

Specific examples of ionic drugs that can be ionized into positive ions and are applicable to iontophoresis include anesthetics (procaine hydrochloride, lidocaine hydrochloride, etc,) gastrointestinal disease therapeutic agents (carnitine chloride, etc,) skeletal muscle relaxants (pancuronium bromide, etc,) and antibiotics (tetracycline preparation, kanamycin preparation, gentamycin preparation, etc.)

Examples of ionic drugs that can be ionized into negative ions include vitamins (vitamin B2, vitamin B12, vitamin C, vitamin E, etc,) adrenocortical hormones (hydrocortisone aqueous preparation, dexamethasone aqueous preparation, predonisolone aqueous preparation, etc,) and antibiotics (penicillin aqueous preparation, chloram phenicol aqueous preparation, etc.)

Furthermore, combinations of ionic drugs may be suitably selected depending upon the disease type, patient condition, and the like. One preferable example of a combination of such ionic drugs is a vaccine and an adjuvant.

Examples of the vaccine in one embodiment of the present invention include BCG vaccine, hepatitis A vaccine, melanoma vaccine, measles vaccine, polio vaccine, and influenza vaccine.

Furthermore, examples of the adjuvant include Monophosphoryl lipid A (MPL), dimyristoylphosphatidylcholine (DMPC), QS-21, Dimethyldioctadecyl ammonium chloride (DDA), and RC-529.

Furthermore, examples of a preferable combination of vaccine and an adjuvant include positively ionized vaccine and RC-529, negatively ionized vaccine and DDA, BCG vaccine and MPL, hepatitis A vaccine and DMPC, and melanoma vaccine and QS-21.

Other combinations of ionic drugs may also be used, in addition to the combinations of vaccine and adjuvant described above. Examples include combinations of a hypotensive drug and a hypotensive diuretic agent, such as lisinopril and hydrochlorothiazide, methyldopa and hydrochlorothiazide, clonidine hydrochloride and chlorthalidone, and benazepryl hydrochloride and hydrochlorothiazide. An example of a combination of diabetic agents includes glyburide and Metformin. Other examples of combination of ionic drugs includes ozagrel hydrochloride and ozagrel sodium, and codeine hydrochloride and promethazine hydrochloride.

An inactive electrode made of a conductive material such as carbon or platinum may be used as the electrode of the electrode structure.

The electrolyte solution holding portion may be formed of a thin film impregnated with an electrolyte solution. The thin film may be formed from the same type of material as that used for the drug holding portion impregnated with an ionic drug, described later.

Electrolyte solutions may be chosen according to a drug to be delivered or the like. Solutions that have an adverse effect on the skin of a living body due to reaction with an electrode may be avoided. Organic acids and salts thereof that are present in the metabolic cycle of a living body may be preferable electrolyte solutions in terms of the biocompatibility. For example, lactic acid and fumaric acid are preferable. Specifically, an aqueous solution comprising 1 M of lactic acid and 1 M of sodium fumarate (1:1) is preferable. Such an electrolyte solution is preferable for the following reasons: the solution has high solubility with respect to water, passes current well, and has low solution electrical resistance when constant current is flowing therethrough. Changes in pH in the power source device are relatively small.

It is preferable to use a cation exchange membrane and an anion exchange membrane in the electrode structure.

Furthermore, the drug holding portion may comprise a thin film impregnated with an ionic drug. When used, such a thin film should be capable of being sufficiently impregnated with an ionic drug. The thin film should also have sufficient characteristics (ion transferability, ion conductivity) allow the ionic drug to migrate to the skin under a predetermined electric field. Examples of materials having satisfactory impregnation characteristics and satisfactory ion transferability characteristics include acrylic resin hydrogels (acrylhydrogel films), segmented polyurethane gel films, and ion conductive porous sheets that form gel solid electrolytes.

No specific limitations are placed on the material or materials used for the package when a plurality of electrode structures are integrated in one package to configure a drug administration unit. An example material is polyolefin for medical equipment. Materials that influence drug delivery are not preferable.

The details of the respective constituent materials are described in WO 03/037425 A1 by the present applicant, which is incorporated herein by reference in its entirety.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

The above description of illustrated embodiments, including what is described in the Abstract, is not intended to be exhaustive or to limit the invention to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to other medical devices, not necessarily the exemplary iontophoresis device generally described above.

1. An iontophoresis device comprising:
a power source device;
a drug administration unit coupled to the power source device and comprising at least a first electrode structure and a second electrode structure that each hold an ionic drug; and
a current control unit that individually controls currents flowing to the first and the second electrode structures,
wherein a predetermined amount of the ionic drug is released from each of the first and the second electrode structures to be administered transdermally to a living body in a predetermined period of time according to a current flowing from the current control unit.

2. An iontophoresis device according to claim 1, wherein the drug administration unit further comprises at least a third electrode structure that does not hold an ionic drug as a counter electrode to the first and the second electrode structures.

3. An iontophoresis device according to claim 2, wherein:
the first electrode structure comprises:
an electrode having the same polarity as that of a drug component of the ionic drug in the electrode structure, the electrode being connected to the power source device;
an electrolyte solution holding portion impregnated with an electrolyte solution, the electrolyte solution holding portion being placed adjacent to the electrode;
an ion exchange membrane that selectively passes ions having a polarity opposite to that of a charged ion of the ionic drug, the ion exchange membrane being placed adjacent to the electrolyte solution holding portion;
a drug holding portion impregnated with the ionic drug, the drug holding portion being placed adjacent to the ion exchange membrane; and
an ion exchange membrane that selectively passes ions having the same polarity as that of a charged ion of the ionic drug, the ion exchange membrane being placed adjacent to the drug holding portion; and
the third electrode structure comprises:
an electrode having a polarity opposite to that of the electrode of the first electrode structure;
an electrolyte solution holding portion impregnated with an electrolyte solution, the electrolyte solution holding portion being placed adjacent to the electrode; and
an ion exchange membrane that selectively passes ions having a polarity opposite to that of a charged ion of the ionic drug in the first electrode structure, the ion exchange membrane being placed adjacent to the electrolyte solution holding portion.

4. An iontophoresis device according to claim 2, wherein:
the first electrode structure comprises:
an electrode having the same polarity as that of a drug component of the ionic drug in the electrode structure, the electrode being connected to the power source device;
an electrolyte solution holding portion impregnated with an electrolyte solution, the electrolyte solution holding portion being placed adjacent to the electrode;
an ion exchange membrane that selectively passes ions having a polarity opposite to that of a charged ion of the ionic drug, the ion exchange membrane being placed adjacent to the electrolyte solution holding portion;
a drug holding portion impregnated with the ionic drug, the drug holding portion being placed adjacent to the ion exchange membrane; and
an ion exchange membrane that selectively passes ions having the same polarity as that of a charged ion of the ionic drug, the ion exchange membrane being placed adjacent to the drug holding portion; and wherein
the third electrode structure comprises:
an electrode having a polarity opposite to that of the electrode in the first electrode structure;
an electrolyte solution holding portion impregnated with an electrolyte solution, the electrolyte solution holding portion being placed adjacent to the electrode;
an ion exchange membrane that selectively passes ions having the same polarity as that of a charged ion of the ionic drug in the first electrode structure, the ion exchange membrane being placed adjacent to the electrolyte solution holding portion;
an electrolyte solution holding portion impregnated with an electrolyte solution, the electrolyte solution holding portion being placed adjacent to the ion exchange membrane; and
an ion exchange membrane that selectively passes ions having a polarity opposite to that of a charged ion of the ionic drug in the first electrode structure, the ion exchange membrane being placed adjacent to the electrolyte solution holding portion.

5. An iontophoresis device according to claim 2, wherein:
the first electrode structure comprises:
an electrode having the same polarity as that of a drug component of the ionic drug in the electrode structure, the electrode being connected to the power source device;
an electrolyte solution holding portion impregnated with an electrolyte solution, the electrolyte solution holding portion being placed adjacent to the electrode;
an ion exchange membrane that selectively passes ions having a polarity opposite to that of a charged ion of the ionic drug in the first electrode structure, the ion exchange membrane being placed adjacent to the electrolyte solution holding portion;
a drug holding portion impregnated with the ionic drug, the drug holding portion being placed adjacent to the ion exchange membrane; and
an ion exchange membrane that selectively passes ions having the same polarity as that of a charged ion of the ionic drug in the first electrode structure, the ion exchange membrane being placed adjacent to the drug holding portion; and
the third electrode structure comprises:
an electrode having a polarity opposite to that of the electrode of the first electrode structure;
an electrolyte solution holding portion impregnated with an electrolyte solution, the electrolyte solution holding portion being placed adjacent to the electrode;
an ion exchange membrane that selectively passes ions having a polarity opposite to that of a charged ion of the ionic drug in the second electrode structure, the ion exchange membrane being placed adjacent to the electrolyte solution holding portion;
a drug holding portion impregnated with an ionic drug, the drug holding portion being placed adjacent to the ion exchange membrane; and
an ion exchange membrane that selectively passes ions having the same polarity as that of a charged ion of the ionic drug in the second electrode structure, the ion exchange membrane being placed adjacent to the drug holding portion.

6. An iontophoresis device according to claim 1, wherein the drug administration unit is configured integrally.

7. An iontophoresis device according to claim 1, wherein the current control unit comprises a load resistor provided between the electrode structure and the power source device, a current detecting portion that detects a current flowing to the load resistor, and a feedback control portion that allows a controlled current to flow to the electrode structure.

8. A method of operating the iontophoresis comprising a power source device, a drug administration unit coupled to the power source device and comprising at least a first and a second electrode structure that each hold an ionic drug, and a control unit that individually controls a current flow to each of the electrode structures, the method comprising:
placing the drug administration unit on a skin surface of a living body;
passing a current through the drug administration unit from the power source device;
individually controlling currents flowing to the electrode structures with the current control unit; and
releasing a predetermined amount of the ionic drug from each of the electrode structures in a predetermined period of time.
